(19) United States
(12) Patent Application Publication
(10) Pub. No.: US 2004/0137053 A1
(43) Pub. Date: Jul. 15, 2004

(54) Method for making water soluble a poorly water soluble antiseptic

(76) Inventors: Dominique Toselli, Nice (FR); Noel Zakarian, Marseille (FR)

Correspondence Address: Oblon Spivak McClelland, Maier & Neustadt, Fourth Floor, 1755 Jefferson Davis Highway, Arlington, VA, 22202, US

(21) Appl. No.: 10/469,603

(22) PCT Filed: Feb. 22, 2002
(86) PCT No.: PCT/FR02/00663

(30) Foreign Application Priority Data
Mar. 1, 2001 (FR) 01/02794

(51) Int. Cl. A61K 9/20

(52) U.S. Cl. 424/465; 264/109

(57) ABSTRACT

The invention relates to a method for preparing water-soluble granules containing at least one naturally poorly water-soluble or -insoluble antiseptic. The invention also relates to the use of said method, in particular for the preparation of pharmaceutical compositions in dry form which can be used to produce, extemporaneously, solutions for mouthwashes.

The invention relates to a process for preparation of water-soluble granules containing at least one antiseptic which is naturally not very soluble or is not soluble in water, and to uses of this process, and especially for the preparation of pharmaceutical compositions occurring in dry form and allowing solutions for mouth baths to be produced extemporaneously.

It is customary for doctors and dentists to prescribe, in the case of disorders of the buccal cavity (mouth ulcers, gingivitis, superficial parodontitis, . . . ) or of the oropharynx, as well as in the operating suites in stomatology and parodontology, mouth baths or gargles which must be carried out several times per day.

The solutions for mouth baths commercially available to date (HEXTRIL®, CORSODYL®, COLLUNOVAR®, ELUDRIL GIVALEX®, ALODONT®, PREXIDINE®, . . . ) are aqueous ethanolic solutions which are packaged in glass or plastic bottles, with a content ranging from 125 to 500 ml, and which are not very practical to use when traveling considering their bulk and the risks of breakage and/or of staining in the case of poor closing of the bottle. Most often, poor compliance with the treatment by the patients results from this, who avoid traveling with their bottle of solutions for mouth baths.

It would therefore be desirable, in order to allow patients to follow their treatment better, to offer them compositions being present in dry form, preferably packaged in unit doses, easily transportable, and able to give, after dispersion in an appropriate volume of water, solutions for mouth baths.

However, it is found that the majority of active principles with an antiseptic aim capable of being used in solutions for mouth baths, such as hexetidine and its derivatives, chlorhexidine and a good number of its salts, chlorobutanol hemihydrate and eugenol, are soluble in organic solvents but are not soluble or are only very sparingly soluble in water. Also, their dispersion in water leads to the formation of a cloudy suspension, with a not very attractive appearance, in which the active principle is not uniformly distributed, which can be manifested in a decrease in the therapeutic efficacy of the mouth baths.

In order to obtain a satisfactory solubilization of these substances, it is expedient to use an organic solvent, ethanol being the best adapted to pharmaceutical use. However, for obvious reasons of public health and also of convenience, it is not conceivable to propose compositions for mouth baths which have to be reconstituted, partially or totally, in ethanol.

Effervescent tablets intended to allow the extemporaneous preparation of solutions for mouth baths have long been known. Such tablets are described, for example, in American U.S. Pat. Nos. 3,772,431, 3,888,976 and 4,919,918. These are in all cases products known to the general public, whose purpose is more to allow cleaning of the buccal cavity and of the teeth, by an effervescent, even foaming, effect, than a disinfection. Therefore, in these patents, the presence of an antiseptic such as cetyldimethylbenzylammonium chloride or chlorhexidine is envisaged only in a very secondary manner. It is to be noted that American U.S. Pat. No. 4,919,918 proposes, in order to ensure solubilization in water of substances which are not very soluble or are not soluble and which may be present in the tablets, to add a surface-active agent of the sodium laurylsulfate, n-laurylsarcosinate or alkylsulfoacetate type, which contributes to the foaming effect sought. Such a foaming effect is not, however, desirable for mouth baths with a therapeutic aim.

Processes allowing active principles which are naturally not very soluble or are not soluble in water to be rendered water-soluble have moreover already been proposed.

Thus, the patent application FR-A-2 649 611 proposes a process for preparation of a therapeutic composition based on acetylsalicylic acid consisting of dissolving this active principle in a solvent and then impregnating a carbohydrate food support with the solution thus formed and then causing the evaporation of the solvent in order to lead to the formation of microparticles or of microcrystals of the active principle, the development of the crystals being limited by the support matrix.

Moreover, the patent application FR-A-2 613 223 proposes a process for preparation of a solid galenic form being present in the form of water-soluble granules containing ginkgo biloba. According to this process, this active principle is solubilized in a solvent in the presence of a surface-active agent, then this solution is next mixed with a water-soluble support which is next subjected to granulation. According to this document, the water-soluble support can equally well be chosen from sucrose, lactose, mannitol and maltodextrins.

However, although these processes allow the solubility of these particular active principles, which are acetyl-salicylic acid and ginkgo biloba, to be improved whatever the nature of the water-soluble support used, they do not entirely give satisfaction when they are applied in order to solubilize antiseptic active principles.

The inventors are, therefore, settled on the aim of providing a process which allows an antiseptic which is naturally not very soluble or is not soluble in water to be rendered water-soluble, in such a way as to be in a position to prepare pharmaceutical compositions which are present in dry form and which, although containing one or more antiseptics which are not very water-soluble or are not water-soluble, are capable of forming homogeneous and stable solutions (without any phase separation) after dispersion in water.

The inventors are, in addition, settled on the aim of providing a process which, while having these advantages, is simple to employ and does not necessitate apparatuses other than those with which pharmaceutical laboratories are conventionally equipped.

The invention accordingly provides a process for preparation of water-soluble granules containing at least one antiseptic, comprising:

a) a stage of wet granulation or impregnation of a pharmaceutically acceptable water-soluble support in the form of granules by means of a solvent containing at least one active principle, and

b) a stage of drying of the granules, characterized by the fact that the water-soluble support in the form of granules is mannitol and that the active principle is an antiseptic.

Within the context of their work, the inventors have in fact noted that, in a surprising manner, when mannitol is subjected to a wet granulation or to an impregnation, by means of a solvent containing an antiseptic which is naturally not very soluble or is not soluble in water, this antiseptic is bound to the mannitol without being denatured, and remains bound to the latter when the granules are subsequently dispersed in water. A solution in which the antiseptic is perfectly dissolved and suitable to exercise its therapeutic effects is thus obtained, as though its solubilization was made possible by the very hydrophilic property of the mannitol to which it is bound.

The inventors have not yet identified the nature of the binding which is established between the antiseptic active principle and the mannitol in the course of the granulation. On the other hand, the existence of this binding has been clearly demonstrated by tests showing that, if a solution obtained by dispersion of granules in water is subjected to filtrations and extractions, the antiseptic active principle is constantly recovered in the fraction where the mannitol is found, without any trace of antiseptic not bound to the mannitol being detected.

Conversely, when xylitol or sorbitol is subjected to a wet granulation or to an impregnation by means of a solvent containing the same antiseptic, then to the dispersion of the granules obtained in water, it is noted that the antiseptic does not remain completely bound to the latter when the granules are subsequently dispersed in water.

According to an advantageous embodiment of this process, the solvent used for the granulation or the impregnation is water, an alcohol or a water/alcohol mixture. The inventors have, in fact, noted that it is not necessary in order that the antiseptic is bound suitably to the mannitol that it is dissolved in the solvent, so that the latter can equally be water in which the antiseptic is found in suspension as an alcohol or a water/alcohol mixture in which it is found in solution.

When the solvent is an alcohol or a water/alcohol mixture, ethanol is preferably used. However, other alcohols such as isopropanol are likewise capable of being used for carrying out the process according to the invention.

According to another advantageous embodiment of the process according to the invention, the antiseptic is chosen from quaternary ammonium, hexetidine, bisbiguanides, chlorhexidine and its salts such as chlorhexidine hydrochloride or acetate, chlorobutanol hemihydrate, eugenol, eucalyptol, tyrothricin and the essential oils such as the essential oil of peppermint.

According to the invention, this antiseptic can be combined with one or more other active principles, antiseptic or nonantiseptic, as a function of the therapeutic effect sought, which can likewise have a low or zero solubility in water, or be perfectly soluble in water, as is especially the case of chlorhexidine digluconate, choline salicylate and of certain quaternary ammonium known to have a good water solubility (cetylpyridinium chloride, for example) which are readily combined with chlorobutanol hemihydrate and hexetidine.

When the active principles which it is desired to combine have very different solubilities, it is preferable to dissolve them in two different solvents (for example an alcohol for the least soluble active principle, water or a water/alcohol mixture for the most soluble active principle) and to carry out the granulation of the mannitol by wetting the latter firstly with the solvent containing the least soluble active principle, then with the solvent containing the most soluble active principle.

Moreover, the solvent used for the granulation or the impregnation of the mannitol can comprise, in addition, one or more excipients such as a colorant (amaranth, cochineal red, Patent Blue V, . . . ) chosen for its compatibility with the antiseptic used and the possible additional active principle(s) and intended to confer a pleasant color to the solution resulting from the dispersion of the granules in water, or any other excipient which it is desired to combine with the antiseptic and which also has a low or zero solubility in water.

According to still another advantageous embodiment of the process according to the invention, the granulation or the impregnation is carried out with a quantity of solvent corresponding to 10 to 80% and, preferably, to 15 to 75% by weight of the weight of mannitol, as long as this solvent has a content by weight of antiseptic of between 10 and 80% and, preferably, between 15 and 70%. Thus, at the end of the drying of the granules, the latter advantageously have a ratio by weight between the mannitol and the antiseptic of between 0.5 and 50 and, preferably, between 1 and 30.

According to the invention, the granulation of the mannitol and the drying of the granules are, preferably, carried out in a fluidized air bed at a temperature which can vary between 20 and 60° C. according to the degree of sensitivity to heat of the antiseptic(s) and of the additional active principle(s) optionally used. However, it is likewise possible to carry out the granulation of the mannitol in a planetary mixer and only to utilize a fluidized air bed for the drying of the granules. This drying can likewise be carried out with the aid of an oven.

According to still another advantageous embodiment of the process according to the invention, it comprises, subsequently to the drying of the granules, an operation of calibration of these granules, for example by screening on a grid, this calibration being, preferably, conducted so as to eliminate all the granules having a size of greater than 5 mm.

The process which has been described has numerous advantages. In fact, by making it possible to render soluble in water antiseptics which are naturally not soluble or not very soluble, it offers the possibility of producing, starting from antiseptics with low or zero water solubility, compositions which are present in dry form and able to form homogeneous and stable solutions after dispersion in water. Moreover, this process is simple to carry out and only uses starting materials (mannitol, water, alcohol) which are easily available and of a cost compatible with exploitation on an industrial scale. Finally, it does not necessitate specific equipment and can be carried out by means of apparatuses with which pharmaceutical laboratories are conventionally equipped, the wet granulation and the impregnation in fact being commonly used for the manufacture of proposed medicaments in capsules and in tablets.

Provided by the invention likewise is the use of a process as defined above for the preparation of a pharmaceutical composition which is present in dry form and able extemporaneously to give a solution for mouth baths after dispersion in water.

Thus the invention likewise provides a pharmaceutical composition which is present in dry form and allows a solution for mouth baths to be produced in an extemporaneous manner by dispersion in water, which is characterized in that it comprises granules formed of mannitol bound to at least one antiseptic.

According to a preferred embodiment of this composition, the antiseptic is chosen from quaternary ammonium, hexetidine, bisbiguanides, chlorhexidine and its salts such as chlorhexidine hydrochloride or acetate, chlorobutanol hemihydrate, eugenol, eucalyptol, tyrothricin and the essential oils such as that of peppermint.

In a particularly preferred manner, the antiseptic is chosen from hexetidine and chlorobutanol hemihydrate, possibly in combination with chlorhexidine digluconate.

According to the invention, the composition can be formed uniquely by granules such as those obtained by the process described above.

However, although mannitol is in itself a sweetener and contributes to giving to the composition a sweetness and a coolness in the mouth at the same time, it will most often be preferred to mix the granules with one or more sweeteners such as sodium cyclamate, sodium saccharinate, aspartame or ammonium glycerinate, and/or one or more flavorings (mint, aniseed, lemon, raspberry, . . . ) in order to make it even more pleasant, both at the level of the taste and of its scent, especially in the case where the antiseptic used does not have very advantageous organoleptic qualities.

Moreover, the composition can be present in the form of a powder resulting from the mixing of the granules with the excipient(s), or in the form of tablets. In the latter case, the composition again comprises one or more excipients chosen from those conventionally used for the compression of powders or granules as flow agents, filling agents and lubricants.

According to another preferred embodiment of the composition according to the invention, the latter is packaged in unit doses each corresponding to one mouth bath. In a particularly preferred manner, each unit dose is packaged in a packing which is air- and watertight, such as, for example, a sachet formed from an aluminum foil lined with a polyethylene film, and whose dimensions are chosen in such a way as to be able to contain, once opened, a volume of water sufficient to make one mouth bath, which thus obviates the use of a glass which is not always to hand, especially if traveling.

The composition according to the invention has numerous advantages. In fact, it disperses easily in water to form a solution which is stable, clear, homogeneous and of a very agreeable taste, even though it contains an antiseptic with not very advantageous organoleptic properties. Being in a dry form, it suppresses the risks of degradation of antiseptics having a tendency to be unstable in solution, as is principally the case with chlorhexidine, whose stability depends closely on the degree of alcohol and the pH. Presented in unit doses, it guarantees the use of an efficacious dose of antiseptic during each mouth bath. Finally, by means of its presentation and its ease of use, it offers the possibility of making a mouth bath under any circumstance without having to travel with a bottle, which is liable to break or to open in an inopportune manner.

The invention further provides a solution for mouth baths, characterized in that it is obtained by dispersing in water a pharmaceutical composition such as defined previously.

The invention will be better understood with the aid of the remainder of the description which follows, which refers to examples of preparation of dry compositions intended to be used for the extemporaneous production of solutions for mouth baths and for demonstration of the ability of these compositions to form homogeneous and stable solutions after dispersion in water.

It goes without saying, however, that this remainder of the description is only given by way of illustrations of the invention and does not in any manner form a limitation.

### EXAMPLE 1

### Composition Comprising Hexetidine as Active Principle

100,000 sachets are produced each containing 540.54 mg of a composition being present in the form of granules and having the following formulation:

Per sachet

| | | |
|---|---|---|
| Hexetidine | 5.000 | mg |
| Amaranth colorant E123 | 0.540 | mg |
| Granulated mannitol | 495.000 | mg |
| Micronized aspartame | 8.000 | mg |
| Mint flavoring | 32.000 | mg |

by a process comprising:

the dissolution of 525 g of hexetidine and of 56.7 g of amaranth E123 in 8.84 liters of ethanol;

the fluidization for 15 minutes of 51.975 kg of granulated mannitol in a fluidized air bed having a temperature of 40° C.;

the spraying of the granulated mannitol with the ethanolic solution of hexetidine and of amaranth, the temperature of the fluidized air being maintained at 40° C.;

the drying of the mannitol thus sprayed in the fluidized air bed until the obtainment of a relative humidity level of less than 2%;

the screening of the resulting granules on a grid having meshes of diameter 2 mm;

the mixing for 3 minutes of these granules with 840 g of micronized aspartame and 3.360 kg of a mint flavoring in a flexible mixer (speed: 30 rpm); and

the putting into sachets of the resulting mixture.

### EXAMPLE 2

### Composition Comprising Chlorhexidine Di-gluconate and Chlorobutanol Hemihydrate

100,000 sachets each containing 590.171 mg of a composition being present in the form of granules and having the following formulation are prepared:

Per sachet

| | | |
|---|---|---|
| 20% chlorhexidine digluconate | 90.000 | mg |
| (or 18.000 mg of chlorhexidine base) | | |
| Chlorobutanol hemihydrate | 90.000 | mg |
| Ponceau 4R colorant E124 | 0.171 | mg |
| Granulated mannitol | 432.000 | mg |
| Micronized aspartame | 25.000 | mg |
| Mint flavoring | 25.000 | mg |

by a process comprising:

the dissolution of 9.45 kg of chlorobutanol hemihydrate in 4.54 liters of ethanol;

the dissolution of 17.955 g of Ponceau colorant in 9.45 kg of a 20% aqueous solution of chlorhexidine gluconate;

the fluidization for 15 minutes of 45.36 kg of granulated mannitol in a fluidized air bed having a temperature of 40° C.;

the spraying of the granulated mannitol with the ethanolic solution of chlorobutanol hydrate, then with the solution of chlorhexidine digluconate and of Ponceau colorant, the temperature of the fluidized air being maintained at 40° C.;

the drying of the mannitol thus sprayed in the fluidized air bed, until the obtainment of a relative humidity level of less than 2%;

the screening of the resulting granules on a grid having meshes of diameter 2 mm;

the mixing for 3 minutes of these granules with 2.625 kg of micronized aspartame and 2.625 kg of a mint flavoring in a flexible mixer (speed: 30 rpm); and

the putting into sachets of the resulting mixture.

### EXAMPLE 3

### Capability of the Compositions to Form Homogeneous and Stable Solutions After Dispersion in Water

The capability of the compositions prepared according to examples 1 and 2—and called below compositions 1 and 2—to form homogeneous and stable solutions after dispersion in water was demonstrated, on the one hand, visually in order to verify the absence of phase separation, and, on the other hand, by analyses aiming to measure the diameter of the particles present in the samples obtained by dispersion of these compositions in water.

The samples were prepared by dispersing the contents of a sachet—or 540.54 mg of composition for the composition 1 and 590.171 mg of composition for the composition 2—in 20 ml of water, and the analyses were carried out by means of a laser diffraction granulometer (red He—Ne laser of 2 mW minimum, of 18 mm diameter and of wavelength 633 nm) MASTERSIZER BANC LONG (MALVERN INSTRUMENTS SA) and of an MS1 sampler from the same company.

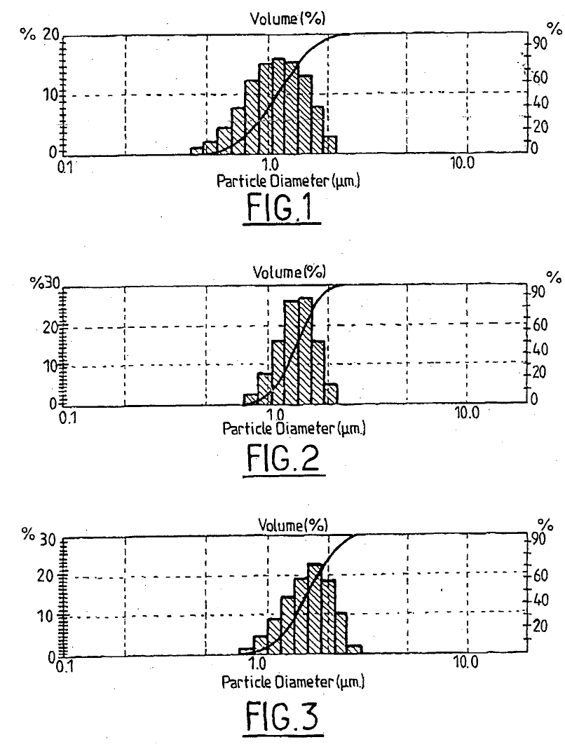

FIGS. 1 to 3 illustrate, at the same time in the form of histograms and of curves, the granulometric distributions obtained respectively for the samples prepared from composition 1 and composition 2, and for an aqueous/ethanolic solution for a reference mouth bath: HEXTRIL®, which has hexetidine as its active principle, like composition 1.

In FIGS. 1 to 3, the axis of the abscissas represents the diameter, expressed in μm, of particles present in the solutions, the axis of the left ordinates represents the percentage of each population of particles—a population corresponding to a hatched rectangle—while the axis of the right ordinates represents the cumulative percentages of the populations of particles.

These figures show that the samples prepared with compositions 1 and 2 and the solution of HEXTRIL® have granulometric distributions which are entirely comparable and that, in all cases, none of the particles detected has a size of greater than 2.28 μm. Especially, the granulometric distribution of the solution of HEXTRIL® is characterized by a peak between 1.68 and 1.95 μm, similar to that observed for the samples obtained starting from composition 1, witnessing to a dissolution of hexetidine in these samples comparable to that observed in the reference specialty.

1. A process for preparation of water-soluble granules containing at least one antiseptic, comprising:
a) a stage of wet granulation or impregnation of a water-soluble pharmaceutically acceptable support in the form of granules by means of a solvent containing at least one active principle, and
b) a stage of drying of the granules, characterized by the fact that the water-soluble support in the form of granules is mannitol and that the active principle is an antiseptic.

2. The process as claimed in claim 1, characterized in that the solvent is water, an alcohol or a water/alcohol mixture.

3. The process as claimed in claim 2, characterized in that the alcohol is ethanol.

4. The process as claimed in any one of the preceding claims, characterized in that the antiseptic is chosen from quaternary ammonium, hexetidine, bisbiguanides, chlorhexidine and its salts, chlorobutanol hemihydrate, eugenol, eucalyptol, tyrothricin and the essential oils.

5. The process as claimed in any one of the preceding claims, characterized in that the granulation or the impregnation of the mannitol is carried out with a quantity of solvent corresponding to 10 to 80% and, preferably, to 15 to 75% by weight of the weight of mannitol.

6. The process as claimed in any one of the preceding claims, characterized in that the solvent has a content by weight of antiseptic of between 10 and 80% and, preferably, between 15 and 70%.

7. The process as claimed in any one of the preceding claims, characterized in that, at the end of the drying of the granules, the latter advantageously have a weight ratio between the mannitol and the antiseptic of between 0.5 and 50 and, preferably, between 1 and 30.

8. The process as claimed in any one of the preceding claims, characterized in that the granulation of the mannitol and the drying of the granules are carried out in a fluidized air bed.

9. The process as claimed in any one of the preceding claims, characterized in that it comprises, subsequently to the drying of the granules, an operation of calibration of these granules.

10. The use of a process as claimed in any one of claims 1 to 9, for the preparation of a pharmaceutical composition which is present in dry form and capable of extemporaneously giving a solution for mouth baths, after dispersion in water.

11. A pharmaceutical composition being present in a dry form for producing in an extemporaneous manner a solution for mouth baths, by dispersion in water, characterized in that it comprises granules formed of mannitol bound to at least one antiseptic, said granules being prepared by a process as claimed in any one of claims 1 to 9.

12. The composition as claimed in claim 11, characterized in that the antiseptic is chosen from quaternary ammonium, hexetidine, bisbiguanides, chlorhexidine and its salts, chlorobutanol hemihydrate, eugenol, eucalyptol, tyrothricin, and the essential oils.

13. The composition as claimed in claim 12, characterized in that the antiseptic is chosen from hexetidine and chlorobutanol hemihydrate, optionally in combination with chlorhexidine di-gluconate.

14. The composition as claimed in any one of claims 11 to 13, characterized in that it comprises one or more sweeteners and/or one or more flavorings.

15. The composition as claimed in any one of claims 11 to 14, characterized in that it is present in the form of a powder or of tablets.

16. The composition according to any one of claims 11 to 15, characterized in that the composition is packaged in unit doses each corresponding to one mouth bath.

17. The composition as claimed in claim 16, characterized in that each unit dose is packaged in a water- and airtight packing, and whose dimensions are chosen so as to be able to contain, once opened, a volume of water sufficient for making one mouth bath.

18. A solution for mouth baths, characterized in that it is obtained by dispersing in water a pharmaceutical composition as claimed in any one of claims 11 to 17.
